(19) United States
(12) Patent Application Publication
(10) Pub. No.: US 2012/0276216 A1
(43) Pub. Date: Nov. 1, 2012

(54) Therapeutic Water Treatment

(76) Inventor: Benjamin Johnson, Evergreen, CO (US)

(21) Appl. No.: 12/399,883

(22) Filed: Mar. 6, 2009

(51) Int. Cl. A61K 33/00; A61P 37/08; A61P 1/00; A61P 25/00; A61P 21/00; C01B 5/00; A61P 17/00

(52) U.S. Cl. 424/600; 423/580.1

(57) ABSTRACT

A therapeutic treatment process a source for generating specific frequencies that are able to provide a concentrated specific frequency to individual molecules of a fluid such as water. The specific frequency that is applied to the individual molecules has been determined to cause the molecule to “mimic” the effect of a specific substance or to cause a cell to do so. One such mechanism is the treated molecule acting as an agonist that triggers the receptor of a cell to release the function of the cell. These cell functions may mimic such substances as oxygen, amino acids, antioxidants, as well as substances that help nerves, hormones, bones, muscle, skin, hair, eyes, digestion, sleep and many more. The effects from the substances may include without limitation non-specific effects such as relaxing, energizing, calming, anti-aging and other effects that may benefit widely disparate individuals, as well as therapeutic effects for specific diseases and conditions for specific individuals.

### FIELD OF THE INVENTION

This invention relates to the field of therapeutic beverages and in particular to a treatment for increasing the beneficial therapeutic properties of beverages.

### BACKGROUND OF THE INVENTION

Enhanced beverages are becoming increasingly more popular. These niche products are marketed for purposes beyond hydration. Such enhanced beverages extend beyond mere flavored hydration drinks to drinks that provide additional health benefits. Examples include beverages having additives such as vitamins, minerals, electrolytes to energy boosters, calming ingredients, exotic herbal and plant extracts. However, many of these enhanced beverages include sweeteners and other added caloric ingredients. Additionally, many of these beverages contain ingredients that may be detrimental to some individuals, such as caffeine and other stimulants.

Enhanced water beverages are one type of enhanced beverages. Enhanced water is a relatively new beverage that has recently been introduced into the market. Enhanced water is simply spring water with one or more added nutraceutical. The nutraceuticals added to water are intended to provide a health benefit, such as an energy booster, vitamin, mineral, electrolyte, calming ingredient or other benefits. Enhanced water is a lighter alternative to the energy or sports beverages.

Some of the ingredients that are added to enhanced beverages such as enhanced water include vitamins, minerals, electrolytes, nutrients, herbs, infused oxygen, stimulants such as caffeine and ginseng, and many other nutraceuticals. Flavors are also often added, typically fruit flavors. There are issues presented by the wide variety of ingredients added to these beverages. Primarily, the consumer is often not properly educated on the effects of these ingredients and the potential for health risks from certain ingredients. For example, individuals who may be suffering from untreated high blood pressure may be at risk from beverages containing high amounts of stimulants such as caffeine or guarana. Also, the interaction between ingredients may also have risks to certain individuals.

One “enhanced” water treatment is described in U.S. Published Patent Application 2008/0157791 A1, filed by Mazursky. Water is treated by infusing the water with vibrations having a specific frequency range between 136.10 Hz to 432.10 Hz. The vibrations are provided by Himalayan bowls or Earth gongs. The application describes a cleansing process to the water by this vibrations performed in a clockwise motion around the water. The sound of Om is then applied to the water to imprint the water with that healing vibration. High frequency vibrations are then applied to the water to create a buffer in the water to retain the infused vibrations according to the patent application. It is unclear as to what benefits other than spiritual benefits are obtained.

The scientific community, over the years, has identified positive health effects from different frequencies through a number of applications. One early pioneer, Dr. Raymond Rife, described a mortality oscillation rate at which pathogens, such as cancer, could be treated and destroyed. U.S. Pat. No. 2,460,707, issued to Moray, discloses enveloping a patient with high frequency electrical fields to treat such ailments as tumors, sinus infections, arthritis and other diseased conditions. Other patents, such as U.S. Pat. No. 5,074,035 issued to Guderain disclosing using a frequency signal applied locally to treat snake bite venom; U.S. Pat. No. 5,908,444 disclosing using a Tesla Coil to treat various disease conditions, U.S. Pat. No. 6,217,604 disclosing using an electromagnetic field to treat AIDs patients, and many other patents that disclose using radio frequency and/or electromagnetic fields to treat patients.

There is presently a need for therapeutic treatments that provide health benefits for a number of conditions. The present invention provides a unique treatment for creating a beverage that is able to provide general non-specific benefits as well as specific benefits for treating specific conditions.

### SUMMARY OF THE INVENTION

The present invention provides a unique treatment process for creating beverages that are able to provide benefits for treating specific and non-specific conditions. The present invention, in a preferred embodiment, provides a frequency enhanced beverage, such as water, that provides benefits for specific and general conditions. The water is treated in concentrated doses at a specific frequency selected to cause the water to provide a therapeutic effect on biological components. This provides specific effects for treating a particular condition.

The enhanced water, in a preferred embodiment, is treated with a frequency generator that focuses a specific frequency on the molecules of the water. The frequency is selected to imprint the molecules with a frequency that provides a particular benefit. For example, the frequency may be selected to improve the health of a particular organ or tissue or it may be selected to increase the energy level of the individual or to cause a calming effect on the individual. The frequency is stored by the water molecules and released either through absorption in a topical application or more preferably ingested orally and released internally in the individual.

Uses of the frequency enhanced water ranges from treating general conditions such as increasing energy levels, causing calming effects, aiding sleep, digestion, relaxation and others to treating specific conditions, such as organ health, tissue health, eyes, hair, skin, etc. The frequencies can be selected to promote the health of biological components by adjusting their frequency levels or by causing the treated water molecules to mimic a beneficial agent by acting as an agonist ligand to cause the release of a receptor's activity or function.

These and other features will be evident from the ensuing detailed description of preferred embodiments and from the claims.

### DETAILED DESCRIPTION OF PREFERRED EMBODIMENTS

A preferred embodiment of the present invention is described herein. It is to be expressly understood that the descriptive embodiments are provided herein for explanatory purposes only and are not meant to unduly limit the claimed inventions. The exemplary embodiments describe the present invention in terms of treating molecules in water and other fluids so that the treated molecules provide beneficial effects to biological components of human and animal bodies. It is to be expressly understood that other uses of the method and products of the present invention are within the scope of the present invention, including without limitation, uses with beverages and other fluids and for treating and modifying all types of substances.

The treatment process of a preferred embodiment of the present invention provides a source for generating specific frequencies, such as a radio frequency generator, electromagnetic field generator and other sources, that are able to provide a concentrated specific frequency to individual molecules of a fluid such as water. The specific frequency that is applied to the individual molecules has been determined to cause the molecule to “mimic” the effect of a specific substance or to cause a cell to do so. One such mechanism is the treated molecule acting as an agonist that triggers the receptor of a cell to release the function of the cell. These cell functions may mimic such substances as oxygen, amino acids, antioxidants, as well as substances that help nerves, hormones, bones, muscle, skin, hair, eyes, digestion, sleep and many more. The effects from the substances may include without limitation non-specific effects such as relaxing, energizing, calming, anti-aging and other effects that may benefit widely disparate individuals, as well as therapeutic effects for specific diseases and conditions for specific individuals.

The treatment process may also use combinations of molecules that have been treated with different frequencies. These combined molecules may be formulated to provide an overall benefit not achieved by molecules treated with a single frequency.

The frequencies or combination of frequencies for providing a particular benefit may be determined through the use of a number of processes. One process includes using a Superconducting Quantum Interference Devices that are able to take readings of biological components, such as organs and tissues. These readings may include determining frequencies of healthy biological components as well as the frequencies of unhealthy or stressed biological components. This enables the specific frequency, frequency range and/or combinations of frequencies for treating such biological components back to health. Another technique is to analyze the molecular structure to determine the frequency ranges. Other processes may be used to determine other frequencies for causing desired effects on biological components. For example, effective resonant frequencies have been identified for use to obtain therapeutic responses are disclosed in U.S. Pat. Nos. 3,876,373; 4,524,079; 5,091,152; 5,552,274; 5,981,182; 6,004,257 and 7,280,874, all incorporated herein by reference.

Once a desired frequency, range of frequencies or combinations of frequencies have been determined, this frequency is generated and applied in concentrated doses to the molecules of the fluid. Water and many other fluids are able to be imprinted with frequencies and store these frequencies. These frequencies can be retained in water in relatively high doses, so even if the enhanced water is later diluted, it will still retain its efficacy.

The treated water may also be combined with other water or fluids that have been treated with other frequencies to provide a desired benefit. Also, other non-treated fluids may be added as well. Specific formulations may be determined to create a compound for specific benefits. The formulated combinations may then be used to treat disorders in biological component such as allergies, sleep deprivation, skin care, and many other disorders.

The frequency enhanced water or other beverage can be sold through existing distribution outlets to individuals. Non-specific frequency enhanced beverages may be sold generally with labeling indicating it's effect. Specific effect frequency enhanced beverages may be sold with labeling either generally or through more controlled distribution outlets such as homeopathic outlets.

The frequency enhanced water is consumed orally by the individual user, or in some instances, it may be applied topically. The frequencies of the water or beverage is released into the individual's body through the digestion process and systemic circulation or absorbed into the tissues and organs. The frequency or frequencies of molecules of the treated water then provide a beneficial effect. For example, one theory of this activity is the treated molecule acts as an agonist molecule that emits an electromagnetic signal. This transmitted electromagnetic signal coresonates with a receptor's molecules. This coresonance activates the cell function to create a desired effect.

These effects, depending on the frequencies and the receptor's cell functions, range from providing specific benefits such as treating allergies, and improving skin conditions, disorders of the nerves, muscle, hair, and eyes to improving sleep, digestion and body functions as well as providing relaxing, energizing, calming, anti-aging and other effects. Other beneficial and therapeutic effects are also provided from the use of the claimed invention.

Other uses of the frequency generated fluids beyond the above described exemplary embodiment are considered to be within the scope of the present invention as set forth in the claims.

What is claimed is:

1. A method for treatment of water for therapeutic benefits, said method comprising the steps of:
providing a fluid; and
generating a specific frequency that has been determined to treat a disorder of a biological component; and
treating said fluid with said generated frequency; and
treating the disorder by consumption of said treated fluid.

2. The method of claim 1 wherein said step of providing a fluid includes:
providing water.

3. The method of claim 1 wherein said step of generating a specific frequency that has been determined to treat a disorder of a biological component includes:
determining the specific frequency which will treat the disorder of biological component; and
generating a concentrated frequency onto said molecule at the determined frequency so that said molecule will provide that frequency to the biological component.

4. The method of claim 1 wherein said method further comprises the steps of:
providing a plurality of fluids;
treating each of said plurality of fluids with differing frequencies; and
combining said plurality of fluids in a specific formulation.

5. The method of claim 1 wherein said method further comprises the steps of:
combining said fluid with other products for providing therapeutic benefits.

6. The method of claim 1 wherein said step of generating a specific frequency that has been determined to have a known therapeutic benefit on a biological component includes:
selecting a specific frequency that will act as agonist to cause a particular cell to release a function having a particular benefit to a biological component.

7. A method for treatment of water for therapeutic benefits, said method comprising the steps of:
providing water molecules;
determining a specific frequency that will treat a disorder of a biological component;
generating said specific frequency;
focusing said specific frequency onto said water molecules; and
providing said treated water molecules for consumption by an individual to treat the disorder of a biological component.

8. The method of claim 7 wherein said method further comprises the steps of:
providing a plurality of water molecules;
treating each of said plurality of water molecules with differing frequencies; and
combining said plurality of water molecules in a specific formulation.

9. The method of claim 7 wherein said method further comprises the steps of:
combining said fluid with other products for providing therapeutic benefits.

10. The method of claim 7 wherein said step of generating a specific frequency that has been determined to have a known therapeutic benefit on a biological component includes:
selecting a specific frequency that will act as agonist to cause a particular cell to release a function having a particular benefit to a biological component.

11. An enhanced beverage for treating disorders in biological components, said enhanced beverage comprising:
a fluid;
molecules in said fluid containing a high level of a specific frequency for treating a particular disorder in a biological component.

12. The enhanced beverage of claim 11 wherein said specific frequency includes:
said molecules acting as an agonist by releasing said specific frequency to a cell receptor to cause cell activity that will treat the disorder.

13. The enhanced beverage of claim 11 wherein said enhanced beverage further includes:
a plurality of fluids; and
each of said plurality of fluids include molecules containing high levels of frequencies differing from the frequencies of the others of said fluids.

14. The enhanced beverage of claim 11 wherein said enhanced beverage further includes:
other products that provide beneficial therapeutic effects on biological components.
